(19) United States
(12) Patent Application Publication
(10) Pub. No.: US 2001/0007680 A1
(43) Pub. Date: Jul. 12, 2001

(54) Use of a film coating as taste-masking coating of oral dosage forms

(76) Inventors: Karl Kolter, Limburgerhof (DE); Silke Scheiffele, Grunstadt (DE); Heinz Einig, Neustadt (DE); Roland Bodmeier, Berlin (DE)

Correspondence Address: Messrs. Keil & Weinkauf, 1101 Connecticut Ave., N.W., Washington, DC, 20036, US

(21) Appl. No.: 09/729,460

(22) Filed: Dec. 5, 2000

(30) Foreign Application Priority Data
Dec. 20, 1999 (DE) 19961897.6

(51) Int. Cl. A61K 9/32

(52) U.S. Cl. 424/482

(57) ABSTRACT

The present invention relates to the use of a film coating consisting of

a) polyvinyl acetate

b) hydrophilic additives

c) other conventional coating ingredients

d) and, where appropriate, a physiologically tolerated acid

as taste-masking coating for oral dosage forms, and to a process for producing such dosage forms.

The present invention relates to the use of a film coating comprising polyvinyl acetate, hydrophilic additives from the group of film-forming water-soluble polymers, of water-insoluble, swelling polymers and/or the group of fine-particle dusting agents as taste-masking coating of oral dosage forms, in particular pharmaceutical dosage forms, and to a process for producing such dosage forms.

Oral dosage forms are taken by the patient in the form of solutions, emulsions, suspensions, capsules and tablets, the solid forms having the greatest importance because of their good dosability, packaging, transportability, stability and, finally, the ease of intake. Many medicinal substances have a bitter taste, which is why either contact of the medicinal substance with the mucosa of the mouth and pharynx must be prevented or the bitter taste must be masked.

In the case of solid dosage forms which are swallowed unchewed it is possible for the entire dosage form to be protected, for example by packing in capsules, by application of a coating layer to the tablet, or the production of tablets which are very hard and disintegrate slowly. However, this way of masking the taste cannot be applied to dosage forms which are broken up before or during administration, for example by masticating or dissolving/dispersing in water. Children, elderly people and many other patients have difficulty in taking tablets and capsules which have not been broken up.

Active ingredients for which the dosage does not permit a small, easily swallowed tablet should be provided as a liquid dosage form or as chewable tablets. The latter variant is desirable because liquid dosage forms do not have the advantages described above.

A well known problem with chewable tablets is the emergence of the unpleasant taste of the active ingredient during the chewing process. The taste of the active ingredient can be improved by adding flavorings and sweeteners so that the bitter taste is masked during the chewing process. In order to obtain adequate masking of the active ingredient with flavorings and sweeteners during the chewing process, a high proportion of the substances is often required so that it is possible in this way only to produce tablets with a low active ingredient concentration.

This method of taste masking is used for dosage forms for children in which the active ingredient concentration is low and thus the proportion of flavorings and sweeteners necessary for masking does not make the tablet unusually large. With many active ingredients, for example ibuprofen, adequate masking is not possible with this method because of the dominating taste.

Another possibility for masking the taste of the active ingredient during the chewing process is coating. Active ingredient-containing shaped articles are coated with a taste-masking coating and then compressed to tablets. During the tablet-chewing process, the coating impedes the release of the active ingredient so that no bitter taste is produced. Rapid release of the active ingredient after swallowing of the masticated tablet is necessary in order to avoid a delay in the onset of action.

A coating for producing tablets with delayed release of active ingredient is described in U.S. Pat. No.4,415,547. Pellets are coated with an organic spray solution consisting of a hydrophilic polymer (PVP), a hydrophobic polymer (ethylcellulose) and other conventional coating ingredients and then, with incorporation of other excipients, compressed to tablets.

The patent EP 317274 describes a taste-masking coating based on cellulose acetate or cellulose acetate butyrate and polyvinylpyrrolidone. The polymers are dissolved in organic solvents, with the solids content of the spray solution being between 8 and 10%. The amount which it is necessary to apply for taste masking is stated to be 12-15% by weight.

The patent EP 523847 describes a polymer mixture consisting of methylaminoethyl methacrylate and neutral methacrylic ester and a cellulose ester plus PVP. A 10% strength organic solution is sprayed onto the pharmaceutically active composition to be coated.

A combination of hydrophobic polymer (EA:MMA) and water-insoluble but swelling polymer is employed for taste masking in patent EP 570606. Addition of the water-insoluble polymer has the additional task of reducing the tackiness of the hydrophobic polymer so that adhesion of the coated particles is avoided.

The coating materials listed herein have the disadvantage that they have only very low elasticity. On severe mechanical stress of the coated particles during the tablet compression process or chewing process in the mouth this may lead to the formation of fissures through which active ingredient diffuses during passage through the mouth and gives rise to a bitter taste.

Besides the poor taste masking, many of these film coatings lead to processing problems such as, for example, adhesion of the pellets or crystals.

In addition, most of the polymers already described for taste masking are incorporated into organic solvents. The disadvantages of organic spray solutions are well known to be the high costs, the risks for people and the environment and, last but not least, a residual amount remaining in the drug form.

It is an object of the present invention to provide a polymer for taste masking of oral, in particular pharmaceutical dosage forms, which does not have the disadvantages listed above.

We have found that this object is achieved by using a film coating comprising

a) polyvinyl acetate

b) hydrophilic additives

c) other conventional coating ingredients

d) and, where appropriate, a physiologically tolerated acid.

Coatings based on polyvinyl acetate and hydrophilic additives for taste masking of medicinal substance-containing shaped articles can be applied directly or be subsequently compressed with other conventional tablet excipients to tablets. The polyvinyl acetate dispersions are prepared without organic solvents and thus have the advantage of the high solids content of an aqueous dispersion, which leads to a shorter processing time and thus to considerable savings of energy and time.

Compared with solutions they also have the advantage of the high solids content of the spray suspension, which leads to a shorter processing time and thus to a saving of energy and time.

Because of the great plasticity and thus high stability in relation to mechanical properties, the coating material is ideally suitable for taste-masking coating of medicinal substance-containing shaped articles and subsequent tableting without damage to the coating.

The described coating material shows no tackiness either during the spraying process or during further processing. The compressed coated particles disintegrate during the chewing process and on addition of liquid back to the initial particles.

The far better elasticity of the coating compared with ethylcellulose or other previously described products is advantageous on severe mechanical stress of the coated particles. The great flexibility of the coating means that there is no formation of fissures, either during the compression process or during the tablet-chewing process, through which active ingredient diffuses during passage through the mouth and gives rise to a bitter taste.

Although polyvinyl acetate is insoluble in water, it can easily swell and allow water to permeate. This is a crucial advantage compared with other lipophilic coating polymers which scarcely allow water to permeate and thus greatly delay release of active ingredient. The coating preparations according to the invention make strong taste masking possible with, nevertheless, a rapid release of active ingredient.

The coating material described in the present invention shows no tackiness either during the spraying process or during the further processing. This makes reproducible coating of the particles possible without formation of twins or multiples during the spraying process. Polyvinyl acetate shows excellent spreading characteristics and adheres very well to the core. Further processing of the coated shaped articles is possible without adhesion leading to variations in dose or problems with the uniformity of content. Despite subsequent compression of the coated particles, they disintegrate during the chewing process and on addition of liquid to the initial particles and thus show the advantages of a multiple unit dosage form.

The polyvinyl acetate employed is compatible with soluble and insoluble hydrophilic polymers and forms an extremely stable spray mixture.

This spray suspension is employed for coating active ingredient-containing shaped articles, it being possible to adjust the taste masking and the rate of release by the ratio of polyvinyl acetate dispersion and hydrophilic additive.

The coated particles are subsequently compressed with other conventional tablet excipients. Because of the high mechanical stability of the coating, neither the tableting nor the chewing process leads to damage impairing the required function. The initial particles are obtained again through the chewing process and the addition of liquid.

Since optimal taste masking is achieved by the intact coating, the addition of flavorings and sweeteners in the chewable tablet can often be reduced.

As soon as the tablet is masticated and the coated particles are swallowed, the active ingredient is released either by permeation through the coating or by dissolving the hydrophilic portion out of the coating.

Polyvinyl acetate has no charged or ionizable groups. It is insoluble in water and thus suitable for producing slow-release formuliations with pH-independent release of active ingredient.

The addition of hydrophilic substances surprisingly achieves excellent taste masking with, at the same time, rapid release of active ingredient.

On use of the coating material for taste masking of active ingredients, the hydrophilic portion determines the duration of the taste-masking effect during passage through the mouth. The hydrophilic portion determines the permeability of the film. For this reason, a chewable tablet produced from particles coated with pure polyvinyl acetate will achieve excellent taste masking but will not release the active ingredient sufficiently quickly after swallowing.

The release can be speeded up by hydrophilic additives while, at the same time, retaining the excellent taste masking.

The hydrophilic additive portion must ensure that good taste masking is present while the tablet is masticated in the mouth and, after the swallowing process, rapid release of the active ingredient should take place. The ratio of polyvinyl acetate to hydrophilic additive is between 1:0.1 and 1:0.75, preferably between 1:0.2 and 1:0.5.

The hydrophilic additives usually employed are film-forming, water-soluble polymers, water-insoluble but swelling polymers or fine-particle dusting agents, it further being possible for sugars such as dextrose or sucrose to be added, oligosaccharides or sugar polymers.

Water-soluble film-forming polymers which can be used are poly(vinyllactams), vinyl acetate/vinylpyrrolidone copolymers, polyvinyl alcohols or cellulose derivatives. Examples are povidone, copovidone, polyvinyl alcohol, hydroxypropylcellulose, hydroxypropylmethylcellulose, hydroxyethylcellulose.

The water-insoluble but swelling polymers comprise crosslinked polyvinylpyrrolidones, crosslinked cellulose or cellulose derivatives or crosslinked starch or starch derivatives. Examples thereof are crospovidone, croscarmellose and crosslinked sodium carboxymethyl starch.

Dusting agents which can be used are highly disperse silicas, fine-particle starches or celluloses or fine-particle salts of phosphoric acid. It is frequently advantageous to combine substances from said groups with one another.

Other conventional ingredients may be added to the spray preparation. These include plasticizers in order to adjust the flexibility of the coating. Examples of plasticizers suitable for polyvinyl acetate are propylene glycol, triacetin, triethyl citrate, tributyl acetylcitrate, polyethylene glycols, pyrrolidone. Further ingredients are non-stick agents such as, for example, talc or glycerol monostearate, dyes such as, for example, iron oxides or quinoline yellow, wetting agents such as, for example, sodium lauryl sulfate or Cremophor RH 40, and antifoams such as, for example, simethicone.

Examples of physiologically tolerated acids which can be used are: sulfamic acid, pivalic acid, malonic acid, succinic acid, pimelic acid, fumaric acid, maleic acid, lactic acid, tartaric acid, malic acid, benzoic acid, salicylic acid, 2-phenylpropionic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, napthalenemono- and -disulfonic acid, laurylsulfuric acid, butanedisulfonic acid.

Examples of bases which can be used are sodium or potassium hydroxide, sodium or potassium carbonate.

The addition of acid in the film coating can reduce the solubility of the active ingredients and the permeation through the film coating and, in this way, make an additional contribution to taste masking.

The coating can also, in contrast to the formulations mentioned in other patents, be produced on an aqueous basis. The polyvinyl acetate dispersion is prepared without organic solvent. The hydrophilic additives are first dissolved or suspended in water and then incorporated into the polyvinyl acetate dispersion. The spray suspension produced in this way has a high solids content, which results in a shorter processing time and thus a saving of energy and time. Polyvinyl acetate can be employed as organic solution for water-sensitive active ingredients, in which case methanol or acetone serves as solvent. The hydrophilic additives can also be added directly to the polymer dispersion.

It is possible to use for the taste-masking coating both powdered substances and granules, pellets, crystals and tablets. In order to achieve optimal taste masking as well as rapid release after the swallowing process, it is also possible to produce active ingredient pellets with a large proportion of disintegrant. The compact round shape and the smooth surface of the pellets make a uniform intact film coating possible so that taste masking is ensured during the chewing process. The disintegrant which is incorporated into the pellets where appropriate then ensures rapid disintegration of the particles and thus speedy release of the active ingredient. This combination achieves optimal taste masking during the passage through the mouth and rapid release in the stomach.

The addition of a physiologically tolerated acid or base may enhance the taste masking through formation of a less soluble form of the active ingredient on entry of water. The less soluble form in this case may be the free base or acid of the active ingredient or else a less soluble salt thereof.

Production of the compacted shaped articles from powdered substances takes place by granulation, preferably in a high shear mixer, rotor granulation or extrusion. For rounding off and smoothing the surface, the shaped articles can be rounded off in a spheronizer. A high density and a maximally round shape are of crucial importance for the subsequent compression of the coated shaped articles because shaped articles of high porosity and irregular shape undergo such extensive deformation that the coating is also affected.

The pellets are composed of from 30 to 98%, preferably 50 to 98%, active ingredient, of from 2 to 70%, preferably 2 to 30%, binder, of from 0 to 5%, preferably 0.1 to 1%, emulsifier and, where appropriate, of from 2 to 30%, preferably 2 to 15%, disintegrant, and, where appropriate, from 0 to 30%, preferably 0 to 20%, of a physiologically tolerated acid or base. The data are % by weight.

The active ingredients employed can be food supplements or additives, vitamins, minerals or trace elements, but particularly preferably active pharmaceutical ingredients.

Active pharmaceutical ingredients requiring taste masking mean, for example, acetaminophen, ibuprofen, naproxen, chlorpheniramine, dextromethorphan, acetylsalicylic acid, loperamide, pseudoephedrine, diphenhydramine, famotidine, cimetidine, ranitidine, nizatidine, salts or mixtures thereof.

It is particularly preferred to use the polymer according to the invention for masking the taste of ibuprofen and acetaminophen.

Possible binders are polyvinylpyrrolidone, hydroxypropylcellulose, hydroxypropylmethylcellulose or maltodextrin. Emulsifiers which can be employed are polyethoxylates of a fatty acid or of a vegetable oil, of a fatty alcohol or of a sorbitan fatty acid compound. Salts of alkyl sulfates such as, for example, sodium lauryl sulfate are likewise suitable. Possible disintegrants are crosslinked polyvinylpyrrolidone, croscarmellose or crosslinked sodium carboxymethyl starch.

For producing the coated particles in a fluidized bed it is possible to use both the top spray and the bottom spray (Wurster) process or processes with a rotating fluidized bed. These processes are described both in “Überzogene Darreichungsformen” published by the wissenschaftliche Verlagsgesellschaft Stuttgart, and in “Aqueous Polymeric Coatings for Pharmaceutical Dosage Forms” published by Marcel Dekker, Inc., Coated Pharmaceutical Dosage Forms, CRC Press, Medpharm Scientific Publishers Stuttgart 1998, Pharmaceutical Coating Technology ed. by G. Cole, Taylor and Francis Ltd. 1995, Aqueous Polymeric Coatings for Pharmaceutical Dosage Forms, Marcel Dekker 1997. The spray suspension is sprayed continuously onto the preheated fluidized material. Coating of the particles is also possible both in a Hüttlin Kugelcoater and in a rotogranulator.

Depending on the particle size and shape, it is necessary to apply an amount of from 1 to 25% by weight to achieve taste asking. The size of the required tablet form likewise plays a part. Smaller tablets are swallowed without mastication and thus require a thinner coating layer than do high-dose tablets which are reduced in size by mastication and thus remain in the mouth longer. The exact amount applied must be established experimentally for each active ingredient.

The shaped articles employed for chewable tablets should have an average particle size of less than 1.0 mm, preferably less than 0.5 mm, since this makes the risk of mastication less.

Tableting of the coating shaped articles to give chewable tablets takes place with conventional direct tableting excipients such as, for example, Ludipress, Ludipress LCE, sorbitol, mannitol, dextrose, sucrose, isomalt, microcrystalline cellulose. It is also possible in a conventional way to employ dry binders, flow regulators, disintegrants and lubricants.

The taste masking can be further enhanced by using a physiological acid or base in the tableting mixture. These additions adjust the pH of the saliva during mastication of the tablet to result in a low solubility of the active ingredient in the saliva. Even less active ingredient reaches the mucosa thereby; for example the solubility of ibuprofen in the saliva can be considerably reduced by using citric acid or tartaric acid.

The film coating developed for taste masking can also be used in order, for example, to isolate two active ingredients which show incompatibilities in a dosage form.

In particular cases a two-layer coating may be worthwhile. For this, a first layer with a relatively large content of hydrophilic additive is sprayed onto the core, and then a second one with a reduced content of hydrophilic additive, or even, in an extreme case, without hydrophilic additive. This makes it possible to reduce the total amount of coating while retaining the taste masking.

The production and use of the coating according to the invention is explained in detail in the following Examples.

### EXAMPLES

The Examples detailed below are based on the laboratory scale. The coating processes correspond to the state of the art and are described in textbooks such as, for example, “Überzogene Darreichungsformen” published by the wissenschaftliche Verlagsgesellschaft Stuttgart and in “Aqueous Polymeric Coatings for Pharmaceutical Dosage Forms” published by Marcel Dekker, Inc. The stated ratio of the components a:b:c employed is based on the solids.

The active ingredients and excipients employed are described below:

Acetaminophen granules, Knoll AG, Ludwigshafen, Germany

Acetaminophen crystals, Knoll AG, Ludwigshafen, Germany

Ibuprofen 25, Knoll AG, Ludwigshafen, Germany

Acetylsalicylic acid crystals,

Kollicoat SR 30 D, BASF AG, Ludwigshafen, Germany

Kollidon SR, BASF AG, Ludwigshafen, Germany

Kollidon 30, BASF AG, Ludwigshafen, Germany

Kollidon 90F, BASF AG, Ludwigshafen, Germany

Cremophor RH 40, BASF AG, Ludwigshafen, Germany

Propylene glycol, BASF AG, Ludwigshafen, Germany

Avicel PH 105, Lehmann & Voss, Hamburg, Germany

Pharmacoat 603, Shin-Etsu, Tokyo, Japan

Aerosil, Degussa-Hüls AG, Frankfurt am Main, Germany

Production of coated shaped articles using an aqueous spray suspension

The solid ingredients of the spray formulation are dissolved in water. The plasticizer is introduced and suspended in the polymer solution. This plasticizer solution is introduced with stirring into the aqueous polyvinyl acetate dispersion. The spray suspension can be employed immediately without standing for further periods. The coated particles are produced on the one hand in the fluidized bed, in which case both the top spray and the bottom spray (Wurster) process can be used, and in equipment with a rotating fluidized bed such as the Hüttlin Kugelcoater or the CF coater.

The spray suspension is sprayed continuously onto the preheated fluidized material. The parameter settings are to be found in the respective example tables. Curing of the coated particles is not normally necessary but may in particular cases improve the taste masking.

### Example 1

| Composition | | | Process parameters | |
|---|---|---|---|---|
| | [%] | [g] | Equipment | Aeromatic, Strea 1, Top spray |

| | | | | | | |
|---|---|---|---|---|---|---|
| Kollicoat | 41.65 | 109.34 | Weight of particles | 350 | Inlet air | 60 |
| SR 30 D | | | [g] | | temperature [° C.] | |
| Propylene | 1.25 | 3.28 | Acetaminophen granules | | Outlet air | 35-40 |
| glycol | | | | | temperature [° C.] | |
| Kollidon 30 | 6.25 | 16.41 | a:b:c ratio | 1:0.5:0.1 | Spraying pressure | 1.2 |
| | | | | | [bar] | |
| Deionized | 50.85 | 133.47 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

### Example 2

| Composition | | | Process parameters | | | |
|---|---|---|---|---|---|---|
| | [%] | [g] | Equipment | Aeromatic, Strea 1, Top spray | | |
| Kollicoat SR | 49.38 | 129.63 | Weight of particles | 350 | Inlet air | 60 |
| 30 D | | | [g] | | temperature [° C.] | |
| Propylene | 1.48 | 3.89 | Acetaminophen granules | | Outlet air | 35-40 |
| glycol | | | | | temperature [° C.] | |
| Mowiol 4/88 | 3.70 | 9.72 | a:b:c ratio | 1:0.25:0.1 | Spraying pressure | 1.2 |
| | | | | | [bar] | |
| Deionized | 45.44 | 119.26 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

Mowiol 4/88 is dissolved with heating. Further processing after cooling of the product corresponds to the above description.

### Example 3

| Composition | | | Process parameters | | | |
|---|---|---|---|---|---|---|
| | [%] | [g] | Equipment | Aeromatic, Strea 1, Top spray | | |
| Kollicoat SR | 51.28 | 134.62 | Weight of particles | 350 | Inlet air | 60 |
| 30 D | | | [g] | | temperature [° C.] | |
| Propylene | 1.54 | 4.04 | Acetaminophen granules | | Outlet air | 35-40 |
| glycol | | | | | temperature [° C.] | |
| Aerosil | 3.08 | 8.08 | a:b:c ratio | 1:0.2:0.1 | Spraying pressure | 1.2 |
| | | | | | [bar] | |
| Deionized | 44.10 | 115.76 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

### Example 4

| Composition | | | Process parameters | | | |
|---|---|---|---|---|---|---|
| | [%] | [g] | Equipment | Aeromatic, Strea 1, Top spray | | |
| Kollicoat SR | 49.38 | 129.63 | Weight of particles | 350 | Inlet air | 60 |
| 30 D | | | [g] | | temperature [° C.] | |
| Propylene | 1.48 | 3.89 | Acetaminophen granules | | Outlet air | 35-40 |
| glycol | | | | | temperature [° C.] | |
| Avicel | 3.70 | 9.72 | a:b:c ratio | 1:0.25:0.1 | Spraying pressure | 1.2 |
| PH 105 | | | | | [bar] | |
| Deionized | 45.44 | 119.26 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

### Example 5

| Composition | | | Process parameters | | | |
|---|---|---|---|---|---|---|
| | [%] | [g] | Equipment | Aeromatic, Strea 1, Top spray | | |
| Kollicoat SR | 49.38 | 129.63 | Weight of particles | 350 | Inlet air | 60 |
| 30 D | | | [g] | | temperature [° C.] | |
| Propylene | 1.48 | 3.89 | Acetaminophen granules | | Outlet air | 35-40 |
| glycol | | | | | temperature [° C.] | |
| Kollidon | 3.70 | 9.72 | a:b:c ratio | 1:0.25:0.1 | Spraying pressure | 1.2 |
| CL-M | | | | | [bar] | |
| Deionized | 45.44 | 119.26 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

### Example 6

| Composition | | | Process parameters | | | |
|---|---|---|---|---|---|---|
| | [%] | [g] | Equipment | Aeromatic, Strea 1, Top spray | | |
| Kollicoat SR | 41.65 | 109.34 | Weight of particles | 350 | Inlet air | 60 |
| 30 D | | | [g] | | temperature [° C.] | |
| Propylene | 1.25 | 3.28 | Acetaminophen crystals | | Outlet air | 35-40 |
| glycol | | | | | temperature [° C.] | |
| Kollidon | 6.25 | 16.41 | a:b:c ratio | 1:0.5:0.1 | Spraying pressure | 1.2 |
| 90 F | | | | | [bar] | |
| Deionized | 50.5 | 133.47 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

### Example 7

| Composition | | | Process parameters | | | |
|---|---|---|---|---|---|---|
| | [%] | [g] | Equipment | Aeromatic, Strea 1, Bottom spray | | |
| Kollicoat SR | 49.38 | 129.63 | Weight of particles | 350 | Inlet air | 60 |
| 30 D | | | [g] | | temperature [° C.] | |
| Propylene | 1.48 | 3.89 | Ibuprofen micropellets | | Outlet air | 35-40 |
| glycol | | | | | temperature [° C.] | |
| Kollidon | 3.70 | 9.72 | a:b:c ratio | 1:0.25:0.1 | Spraying pressure | 1.2 |
| 30 | | | | | [bar] | |
| Deionized | 45.44 | 119.26 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |

### Example 8

| Composition | | | Process parameters | | | |
|---|---|---|---|---|---|---|
| | [%] | [g] | Equipment | Aeromatic, Strea 1, Bottom spray | | |
| Kollicoat SR | 47.62 | 125.00 | Weight of particles | 350 | Inlet air | 60 |
| 30 D | | | [g] | | temperature [° C.] | |
| Propylene | 1.43 | 3.75 | Ibuprofen micropellets | | Outlet air | 35-40 |
| glycol | | | | | temperature [° C.] | |
| Kollidon | 0.71 | 1.86 | a:b:c ratio | 1:0.25:0.1 | Spraying pressure | 1.2 |
| 30 | | | | | [bar] | |
| Avicel PH | 3.57 | 9.37 | Amount applied | 15 | Spraying rate | 3-5 |
| 105 | | | [%] | | [g/min] | |
| Deionized | 46.67 | 122.59 | Solids content of | 20 | After-drying | 45° C., |
| water | | | the spray mixture | | [° C., min] | 3 |
| | | | [%] | | | |
| Total | 100.0 | 262.5 | | | | |

### Example 9

| Composition, Coating 1 | | | Process parameters | | | |
|---|---|---|---|---|---|---|
| | [%] | [g] | Equipment | Aeromatic, Strea 1, Bottom spray | | |
| Kollicoat SR | 41.67 | 109.38 | Weight of particles | 350 | Inlet air | 60 |
| 30 D | | | [g] | | temperature [° C.] | |
| Propylene | 1.25 | 3.28 | Ibuprofen micropellets | | Outlet air | 35-40 |
| glycol | | | | | temperature [° C.] | |
| Kollidon 30 | 6.25 | 16.41 | a:b:c ratio | 1:0.25:0.1 | Spraying pressure | 1.2 |
| | | | | | [bar] | |
| Deionized | 50.83 | 133.43 | Amount applied | 15 | Spraying rate | 3-5 |
| water | | | [%] | | [g/min] | |
| Total | 100.0 | 262.5 | Solids content of | 20 | After-drying | 45° C., |
| | | | the spray mixture | | [° C., min] | 3 |
| | | | [%] | | | |
| Composition, Coating 2 | | | Process parameters | | | |
| | [%] | [g] | Equipment | Aeromatic, Strea 1, Bottom spray | | |
| Kollicoat SR | 49.38 | 17.28 | Weight of particles | 350 | Inlet air | 60 |
| 30 D | | | [g] | | temperature [° C.] | |
| Propylene | 1.48 | 0.52 | Ibuprofen micropellets | | Outlet air | 35-40 |
| glycol | | | | | | |
| Kollidon 30 | 3.70 | 1.30 | a:b:c ratio | 1:0.25:0.1 | Spraying pressure | 1.2 |
| | | | | | [bar] | |
| Deionized | 45.44 | 15.9 | Amount applied | 2 | Spraying rate | 3-5 |
| water | | | [%] | | [g/min] | |
| Total | 100.0 | 35.0 | Solids content of | 20 | After-drying | 45° C., |
| | | | the spray mixture | | [° C., min] | 3 |
| | | | [%] | | | |

No bitter or unpleasant taste of the medicinal substance was detectable in any of the Examples.

Production of coated shaped articles using an organic spray suspension

The solid ingredients of the spray formulation are dissolved in acetone or methanol. The plasticizer is introduced and suspended in the polymer solution. The spray suspension can be employed immediately without standing for further periods. The coated particles are produced on the one hand in the fluidized bed, in which case both the top spray and the bottom spray (Wurster) process can be used, and in a Hüttlin Kugelcoater.

The spray suspension is sprayed continuously onto the preheated fluidized material. The parameter settings are to be found in the respective example tables. Curing of the coated particles is unnecessary but after-drying is advisable in order to remove completely residues of the organic solvent.

### Example 10

| Composition | | | Process parameters | | | |
|---|---|---|---|---|---|---|
| | [%] | [g] | Equipment | Aeromatic, Strea 1, Bottom spray | | |
| Kollidon SR | 9.35 | 32.81 | Weight of particles | 350 | Inlet air | 60 |
| | | | [g] | | temperature [° C.] | |
| Propylene | 0.95 | 3.28 | Ibuprofen micropellets | | Outlet air | 35-40 |
| glycol | | | | | temperature [° C.] | |
| Kollidon 30 | 4.70 | 16.41 | a:b:c ratio | 1:0.5:0.1 | Spraying pressure | 1.2 |
| | | | | | [bar] | |
| Acetone | 85.0 | 297.5 | Amount applied | 15 | Spraying rate | 5-7 |
| | | | [%] | | [g/min] | |
| Total | 100.0 | 350.0 | Solids content of | 15 | After-drying | 45° C. |
| | | | the spray mixture | | [° C., min] | 3 |
| | | | [%] | | | |

### Example 11

| Composition | | | Process parameters | | | |
|---|---|---|---|---|---|---|
| | [%] | [g] | Equipment | Aeromatic, Strea 1, Top spray | | |
| Kollidon SR | 11.11 | 38.89 | Weight of particles | 350 | Inlet air | 60 |
| | | | [g] | | temperature [° C.] | |
| Propylene | 1.11 | 3.89 | Acetaminophen granules | | Outlet air | 35-40 |
| glycol | | | | | temperature [° C.] | |
| Avicel PH 105 | 2.78 | 9.72 | a:b:c ratio | 1:0.25:0.1 | Spraying pressure | 1.2 |
| | | | | | [bar] | |
| Methanol | 85.0 | 297.5 | Amount applied | 15 | Spraying rate | 5-7 |
| | | | [%] | | [g/min] | |
| Total | 100.0 | 350.0 | Solids content of | 15 | After-drying | 45° C., |
| | | | the spray mixture | | [° C., min] | 3 |
| | | | [%] | | | |

No bitter or unpleasant taste of the medicinal substances was detectable.

### Example 12

| Composition | | | Process parameters | | | |
|---|---|---|---|---|---|---|
| | [%] | [g] | Equipment | Aeromatic, Strea 1, Top spray | | |
| Kollicoat SR | 9.35 | 32.81 | Weight of particles | 350 | Inlet air | 60 |
| 30 D | | | [g] | | temperature [° C.] | |
| Propylene | 0.95 | 3.28 | Acetylsalicylic acid crystals | | Outlet air | 35-40 |
| glycol | | | | | temperature [° C.] | |
| Kollidon 30 | 4.70 | 16.41 | a:b:c ratio | 1:0.5:0.1 | Spraying pressure | 1.2 |
| | | | | | [bar] | |
| Deionized | 85.0 | 297.5 | Amount applied | 15 | Spraying rate | 3-5 |
| water | | | | [%] | [g/min] | |
| Total | 100.0 | 350.0 | Solids content of | 15 | After-drying | 45° C., |
| | | | the spray mixture | | [° C., min] | 3 |
| | | | [%] | | | |

Production of chewable tablets

The weighed powder ingredients are passed through a sieve with a mesh width of 0.8 mm and then mixed in a Turbula mixer for 10 min. The tableting parameters are adjusted so that adequate hardness with a friability of <1% is achieved.

| | | | Process parameters | |
|---|---|---|---|---|
| Composition | | | | Korsch EKO |
| | [%] | [g] | Equipment | tableting press |
| Coated granules | 40.0 | 600.0 | Punch form: | 16mm biplanar with |
| Ludipress LCE | 59.5 | 892.5 | | bevel |
| Magnesium stearate | 0.5 | 7.5 | Hardness [N] | 50-80N |
| Total | 100.0 | 1500.0 | | |

No bitter or unpleasant taste was detectable on taking the tablets.

We claim:

1. The use of a film coating consisting of
a) polyvinyl acetate
b) hydrophilic additives
c) other conventional coating ingredients
d) and, where appropriate, a physiologically tolerated acid
as taste-masking coating for oral dosage forms.

2. The use of a film coating as claimed in claim 1, wherein the hydrophilic additives are selected from the group of film-forming water-soluble polymers and/or from the group of water-insoluble but swelling polymers and/or from the group of very fine-particle dusting agents.

3. The use of a film coating as claimed in either of claims 1 or 2, wherein the film-forming water-soluble polymers are selected from the group of poly(vinyllactams), vinylpyrrolidone/vinyl acetate copolymers, polyvinyl alcohols or cellulose derivatives, as water-insoluble but highly swelling polymers crosslinked poly(vinyllactams), cellulose or cellulose derivatives or starch derivatives and as fine-particle dusting agents highly disperse silicas, fine-particle starches, fine-particle celluloses or fine-particle salts of phosphoric acid.

4. The use of the film coating as claimed in any of claims 1 to 3, wherein the ratio by weight amounts to
a) 50 to 90% polyvinyl acetate
b) 10 to 75% hydrophilic additives
c) 0 to 20% other conventional coating ingredients
d) and, where appropriate, 0 to 30% of a physiologically tolerated acid.

5. The use of the film coating as claimed in any of claims 1 to 4, wherein the ratio by weight of the coating material a:b is from 1:0.1 to 1:0.75.

6. The use of the film coating as claimed in any of claims 1 to 5, wherein the taste-masking coating comprises 5 to 25% by weight based on the total weight of the coated shaped articles.

7. An oral dosage form with an active ingredient-containing core and a taste-masking coating consisting of
a) polyvinyl acetate
b) hydrophilic additives
c) other conventional coating ingredients
d) and, where appropriate, a physiologically tolerated acid or base.

8. An oral dosage form as claimed in claim 7, which comprises the following substances based on the weight of the core
a) 30 to 98% active ingredient
b) 2 to 70% binder
c) 0.1 to 5.0% emulsifier and, where appropriate,
d) 2 to 30% disintegrant
e) and, where appropriate, 0 to 20% of a physiologically tolerated acid or base.

9. An oral dosage form as claimed in either of claims 7 or 8, which comprises as active ingredients food supplements or additives, vitamins, minerals or trace elements or active pharmaceutical ingredients.

10. An oral dosage form as claimed in any of claims 7 to 9, which comprises active pharmaceutical ingredients as active ingredients.

11. An oral dosage form as claimed in either of claims 7 or 10, which comprises as active ingredient acetaminophen, ibuprofen, naproxen, chlorpheniramine, dextromethorphan, acetylsalicylic acid, loperamide, pseudoephedrine, diphenhydramine, famotidine, cimetidine, ranitidine, nizatidine, salts or combinations thereof.

12. A taste-masked oral dosage form obtainable by compression of at least one preparation as claimed in claims 7 to 11 with conventional tablet excipients.

13. A taste-masked oral dosage form as claimed in claim 12, wherein from 0 to 40% of a physiologically tolerated acid or base are added to the tablet mixture.

14. A process for producing a taste-masked oral dosage form comprising the steps
a) production of medicinal substance-containing shaped articles from active ingredient, binder, disintegrant and emulsifier,
b) coating of medicinal substance-containing shaped articles with the film coating as claimed in claim 1 and
c) compression of the coated medicinal substance with conventional tablet excipients.
